(19) United States
(12) Patent Application Publication
(10) Pub. No.: US 2025/0180541 A1
(43) Pub. Date: Jun. 5, 2025

(54) URINE TEST DEVICE FOR TOILET AND ITS CONTROL METHOD

(71) Applicant: Hak Soo Kim, San Diego, CA (US)

(72) Inventor: Hak Soo Kim, San Diego, CA (US)

(21) Appl. No.: 18/957,846

(22) Filed: Nov. 24, 2024

(30) Foreign Application Priority Data
Dec. 4, 2023 (KR) 10-2023-0173010

(51) Int. Cl. G01N 33/493; E03D 9/00; G01N 21/84; G01N 33/487

(52) U.S. Cl. CPC G01N 33/493; E03D 9/00; G01N 21/8483; G01N 33/4875

(57) ABSTRACT

The present invention relates to a urine test device for toilet, including a housing which is installed inside the toilet and can test urine by staining a urine test strip with urine, wherein said housing receives the urine test strip and moves the urine test strip to the urine collection area through at least one or more motors and rollers, and transmit the measurement results to the user terminal after measuring the color change of the urine test strip stained with urine through at least one or more sensors.

### CROSS-REFERENCE TO RELATED APPLICATIONS

This application claims priority from Korean Patent Application No. 10-2023-0173010, filed on Dec. 4, 2023, the entire contents of which are hereby incorporated by reference.

### TECHNICAL FIELD

The present invention relates to a urine test device for toilet and its control method, and more particularly, to a urine test device for toilet and its control method which is installed on the toilet, automatically collects urine after receiving the input of the user's type and can test the urine by staining a urine test strip with urine and detecting the color change through a sensor.

### BACKGROUND

Urine can sort the risks of various diseases such as endocrine diseases, metabolic diseases and the like as well as urinary tract diseases, through ingredient test. Because it is simpler than other types of tests, urine test is one of the essential tests performed in the examination process of checking the health status of body. As the demand for in vitro diagnostic test increases due to the aging era, daily routine management and changes of personalization, urine test strip and urine test device that were used only for professional use are expanding to personal use. In addition, demand is increasing as the importance of urine test which is a simple and an inexpensive test and provides a lot of clinical data is recognized.

In the urine test strip which is generally sold, If it is stored for a long time, measurement is inaccurate due to the change of its characteristics, and if test is not performed within a certain time after staining the urine test strip with urine, the accuracy significantly decreases. In addition, the results may vary depending on how much urine has come in contact with the urine test strip. For an individual to test urine, the most accurate method is to go to a hospital or urine collection site, transfer urine to a urine container, and send it to an institution with test equipment in order to test urine. However, it has the disadvantage of being expensive and time-consuming. Therefore, there is a need for a urine test device that can be installed on a privately used toilet.

There is Korean Patent Laid-Open Publication No. 10-2022-0086389 (urine test device and method for controlling the same) as prior art. But it only includes a catheter through which urine is injected, an upper barrel including a camera and a door connected to the catheter and divided into a predetermined number of compartments to be rotated in stages in a vertical direction, a test strip disposed on the outer surface of the upper barrel, a lower barrel into which the urine and the test strip fall by rotation of the door, and a control unit configured to move the test strip into the inside of the upper barrel to make test strip contact with the accumulated urine, and rotate the door so that the test strip falls into the lower barrel when the camera photographs the test strip.

### SUMMARY

### Problem to Solve

The problem that the present invention aims to solve is to provide a urine test device for toilet and its control method which can automatically collect urine by receiving the input of user type and urine test start from the user and test urine by staining a urine test strip with the collected urine and detecting the color change through a sensor.

### Means to Solve the Problem

The present invention relates to a urine test device for toilet, comprising a housing installed inside the toilet and capable of testing urine by staining a urine test strip with urine, wherein the housing receives the urine test strip and moves the urine test strip to the urine collection area through at least one or more motors and rollers, and can transmit the measurement results to the user terminal after measuring the color change of the urine test strip stained with urine through at least one or more sensors.

The present invention relates to a control method of a urine test device for toilet, which includes the step that a urine test strip is inserted into the housing when the input/output unit receives the input of the user type and the start of the urine test, the step that motor moves the urine test strip to before the urine collection area by rotating at least one or more rollers when the sensor unit of the housing recognizes the insertion of the urine test strip, the step that the sensor unit of the housing recognizes information about the urine test strip when the urine test strip arrives before the urine collection area, the step that motor of the housing rotates at least one or more rollers to move the urine test strip to the urine collection area, and if the movement of the urine test strip is completed, the cutter of the housing separates the sectors of the urine test strip to complete preparation for urine collection, the step that the housing drops urine on the separated urine test strip after collecting urine, the step that the housing moves the urine test strip stained with urine to a sensor unit outside the urine collection area after a certain time elapsed, the step that the sensor unit of the housing measures the color change of the test area of the urine test strip and judges test completion, the step that if the sensor unit of the housing judges the completion of the test, a motor of the housing rotates at least one or more rollers to discharge the test-completed urine test strip into a collection box of the housing, and the step that the communication unit transmit a color change measured by the sensor unit of the housing to the user terminal based on the user type.

### Effect of Invention

According to the present invention, a urine test device can be installed on a toilet to receive the input of user type and automatically collect urine.

In addition, the urine test strip used by inserting into the urine test device can be supplied through a urine test cartridge.

In addition, urine can be tested by detecting color change of urine test strip stained with urine through a sensor, and the test results can be transmitted to the user terminal.

In addition, when collecting urine, if certain conditions are met based on temperature and humidity sensors, the valve opens and urine can be smeared on the urine test strip.

In addition, when collecting urine, urine is placed in a fixed amount cup, and if a certain condition is met, the fixed amount cup is overturned and urine can be smeared on the urine test strip.

### BRIEF DESCRIPTION OF THE DRAWINGS

FIGS. 1A and 1B are a cross-sectional view and a plan view of a urine test device for toilet according to an embodiment of the present invention.

FIG. 2 is a configuration diagram of the urine test device for toilet according to an embodiment of the present invention.

FIGS. 3A and 3B are plan views of the input and output unit of the urine test device for toilet according to an embodiment of the present invention.

FIG. 4 is a cross-sectional view of the inside of the housing of the urine test device for toilet according to an embodiment of the present invention.

FIG. 5 is a flowchart for explaining the control method of the urine test device for toilet according to an embodiment of the present invention.

FIG. 6 is a flowchart for explaining the valve method urine collection method of the urine test device for toilet according to an embodiment of the present invention.

FIG. 7 is a cross-sectional view of the inside of the housing for explaining the valve method urine collection method of the urine test device for toilet according to an embodiment of the present invention.

FIG. 8 is a flowchart for explaining the fixed amount cup method urine collection method of the urine test device for toilet according to an embodiment of the present invention.

FIGS. 9A and 9B are cross-sectional views of the inside of the housing for explaining the urine collection method using a fixed amount cup method urine collection method of the urine test device for toilet according to an embodiment of the present invention.

### DETAILED DESCRIPTION OF EXEMPLARY EMBODIMENTS

Specific structural or functional descriptions of the embodiments according to the concept of the present invention disclosed in this specification are merely illustrative for the purpose of explaining the embodiments according to the concept of the present invention. The embodiments may be implemented in various forms and are not limited to the embodiments described in this specification.

Since the embodiments according to the concept of the present invention can make various changes and have various forms, the embodiments will be illustrated in the drawings and described in detail in this specification. However, this is not intended to limit the embodiments according to the concept of the present invention to specific disclosed forms, and includes all changes, equivalents or substitutes included in the spirit and technical scope of the present invention.

The terms used in this specification are only used to describe specific embodiments and are not intended to limit the present invention. Singular expressions include plural expressions unless the context clearly indicates otherwise. In this specification, terms such as “comprise” or “include” or “have” are intended to designate the presence of features, numbers, steps, operations, components, parts, or combinations thereof described in this specification, and it should be understood that it does not exclude in advance the possibility of the existence or addition of one or more other features, numbers, steps, operations, components, parts, or combinations thereof.

Hereinafter, embodiments of the present invention will be described in detail with reference to the drawings attached to this specification.

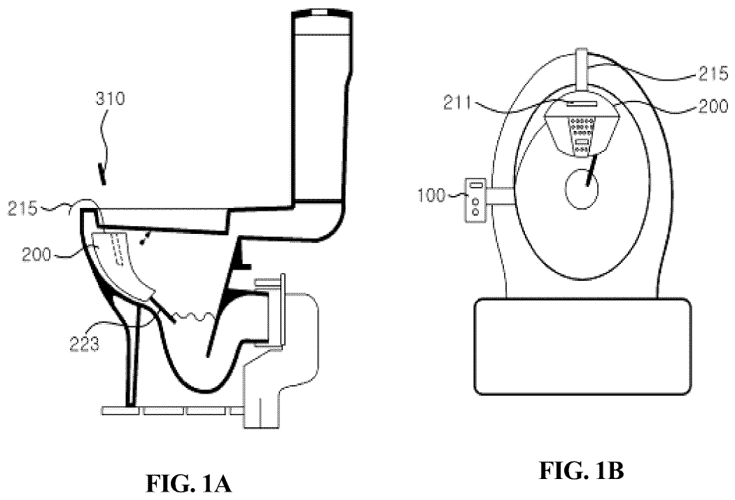

FIG. 1 is a cross-sectional view and a plan view of a urine test device for toilet according to an embodiment of the present invention.

Referring to FIG. 1(a), it shows a cross-sectional view of the urine test device 10 for toilet installed on a toilet. A housing 200 of the urine test device 10 for toilet can be installed inside the toilet through a holder 215, and a urine test can be performed by inserting a urine test strip 310 into the housing 200. In addition, a pump 223 for drawing up water from the water tank of the toilet for cleaning may be located below the housing 200.

Referring to FIG. 1(b), it shows a plan view of the urine test device 10 for toilet installed on the toilet. The housing 200 of the urine test device 10 for toilet may be installed inside the toilet through the holder 215, and an input/output unit 100 may be installed outside the toilet. The housing 200 can insert a urine test cartridge (not shown) through a cartridge input unit 211. The urine test cartridge (not shown) may consist of a urine test strip 310 and a reel around which the urine test strip 310 is wound. In this case, the urine test strip 310 may be in the form of several strips connected. In addition, the urine test strip 310 may be equipped with a protective tape to protect it from other foreign substances, but is not necessarily limited to this. The urine test cartridge (not shown) is inserted into a cartridge input unit 211, and only the urine test strip 310 is inserted into the housing 200, and the reel can be stored in a storage box (not shown) next to the cartridge input unit 211. The storage box (not shown) may be attached to the toilet with it being inclined toward the outside of the toilet so that the urine test strip 310 can be easily released from the reel, but is not necessarily limited to this. The reel of the urine test cartridge (not shown) may have a fixing hole in the middle so that it can rotate well in the storage box (not shown), and it has a mechanism that holds the fixing hole so that the reel does not generate noise when it rotates in the storage box.

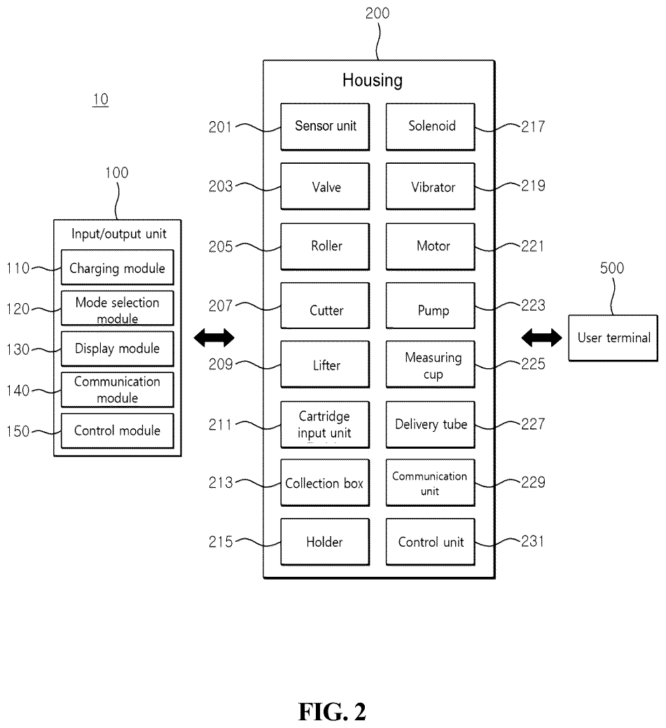

FIG. 2 is a configuration diagram of a urine test device for toilet according to an embodiment of the present invention

Referring to FIG. 2, the urine test device 10 for toilet consists of the input/output unit 100, the housing 200 and a user terminal 500. The input/output unit 100 may be installed outside the toilet, and the housing 200 may be installed inside the toilet, but is not necessarily limited thereto. In addition, the input/output unit 100 and the housing 200 may be electrically connected through a wire (not shown), but are not necessarily limited thereto.

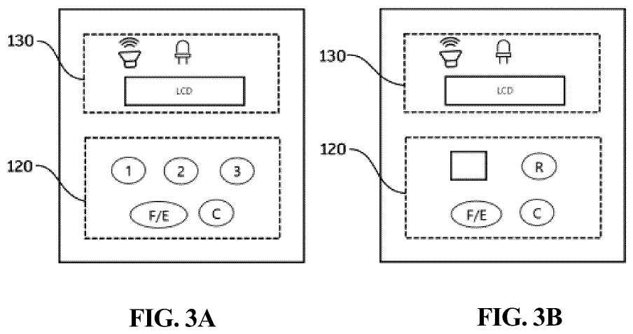

The input/output unit 100 consists of a charging module 110, a mode selection module 120, a display module 130, a communication module 140 and a control module 150. FIG. 3 is a plan view of the input and output unit of the urine test device for toilet according to an embodiment of the present invention, and the input and output unit 100 will be described below with reference to FIG. 3.

The charging module 110 can receive or supply electricity needed to drive the input/output unit 100 and the housing 200. The charging module 110 has a built-in battery to supply electricity, and includes a charging port (not shown) through which electricity for charging the battery can be supplied from the outside. The charging port (not shown) may be at least one of a DC Jack or USB_C type connector, but is not necessarily limited thereto. In addition, the charging module 110 can manage the current charging state of the urine test device for toilet by continuously checking the remaining amount of the built-in battery. In addition, the charging module 110 can maximize the electricity supply limit required for operation when the mode selection module 120 is converted from the energy-saving mode (sleep mode) to the operating mode.

The mode selection module 120 can receive the input of at least one mode of user type, user registration, urine test start and cleaning start from the user. Referring to FIG. 3(a), \textcircled{1}, \textcircled{2}, and \textcircled{3} represent input devices that can select the user type, and F/E is Feed/Eject mode and represents an input device which allows the urine test strip 310 to be inserted into the housing 200. And C is clean mode and represents an input device that allows the housing 200 to be cleaned. The input device capable of inputting the start of a urine test is not shown. The mode selection module 120 may judge as the urine test start when it receives the user type without separately inputting the urine test start mode from the user, but it is not necessarily limited to this. If the mode selection module 120 judges as the urine test start after receiving the input of the user type from the user, it can transmit the start of the urine test to the user terminal 500 of the input user type. The mode selection module 120 can convert from the energy saving mode (sleep mode) to the operating mode when the user type is input based on at least one of fingerprint recognition and an input device capable of selecting a user type mode.

FIG. 3(a) shows only three types of users as \textcircled{1}, \textcircled{2}, and \textcircled{3}, but is not necessarily limited to this. The user type can confirm user information through user registration content previously stored and registered by the user through the application of the user terminal 500. In addition, the mode selection module 120 can receive the input of user type through fingerprint recognition. □ in FIG. 3(b) is an input device that receives the input of a fingerprint from an user. The user inputs a fingerprint through this, and the mode selection module 120 can confirm the user based on the input fingerprint and notify the user terminal 500 of the fingerprint of the start of the urine test. In addition, the mode selection module 120 also may register a user directly at the input/output unit 100 which is not the user terminal 500. Referring to FIG. 3(b), ® is an input device that can start user registration from the user, and the mode selection module 120 can receive the input of user registration start from the user through this. When the mode selection module 120 receives the input of user registration start from the user, it can transmit the notification of a user registration start to the user terminal 500. If the user terminal 500 receives a notification of a user registration start, the user can register the user through fingerprint recognition by placing the user's finger on □. The mode selection module 120 can complete user registration by registering the user's fingerprint and transmit a notification of completion of user registration to the user terminal 500.

In addition, the mode selection module 120 may further include a sound sensor or a microphone (not shown) to recognize the sound of the water going down in the toilet, and can notify the housing 200 of the cleaning start mode after the water of the toilet has gone down.

The display module 130 may display at least one of whether the urine test cartridge is inserted, the remaining amount of the urine test strip 310, whether the urine test device 10 for toilet is broken, and whether the urine test device 10 for toilet is operated. Referring to FIG. 3, a notification may be displayed through at least one of a speaker, a LED, and a LCD when an error occurs or notification is required in at least one of whether a urine test cartridge is inserted, a remaining amount of the urine test strip 310, whether the urine test device 10 for toilet is broken and whether the urine test device 10 for toilet is operated. In one embodiment, when the display module 130 receives a notification indicating that the remaining battery quantity is insufficient from the charging module 110, it can display the phrase “LOW BATTERY” on the LCD. At this time, the LCD of the display module 130 may be an e-ink type LCD that can display information even without a power supply in order to reduce power consumption, but is not necessarily limited to this. In another embodiment, when the display module 130 receives a notification that the battery is being charged from the charging module 110, it may display that charging is in progress through the LED and the LCD, and may display charging completion when the battery charging is completed. In addition, when the display module 130 receives the completion of preparation for urine collection from the housing 200, it can convey the completion of preparation for urine collection to the user using at least one of a speaker, LED and LCD.

The communication module 140 can communicate with the housing 200, the user terminal 500 and each component of the input/output unit 100 through a network. The network may include at least one or more of 3GPP (3rd Generation Partnership Project) network, LTE (Long Term Evolution) network, 5G network, WIMAX (World Interoperability for Microwave Access) network, wired and wireless Internet, LAN (Local Area Network), and Wireless LAN (Wireless Local Area Network), WAN (Wide Area Network), PAN (Personal Area Network), Bluetooth network, WI-FI network, NFC (Near Field Communication) network, satellite broadcasting network, analog broadcasting network and DMB (Digital Multimedia Broadcasting) network, but it is not necessarily limited thereto. In addition, the communication module 140 may use a low-power WIFI module or Bluetooth Low Energy (BLE) Bluetooth Module, IOT cellular module or Zigbee communication to use low power. The control module 150 can control each component of the input/output unit 100.

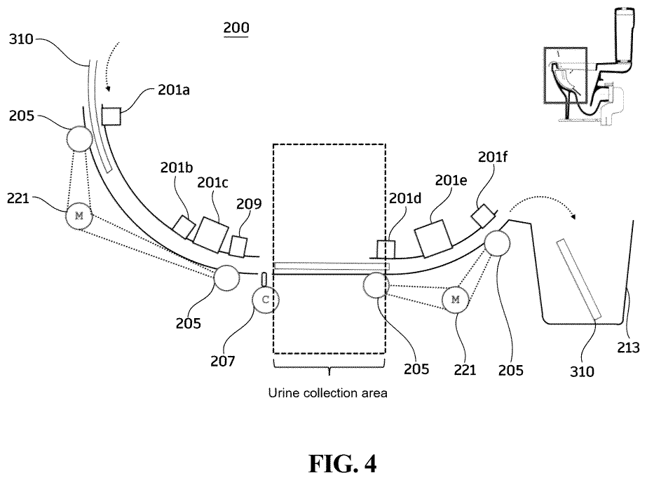

The housing 200 consists of a sensor unit 201, a valve 203, a roller 205, a cutter 207, a lifter 209, a cartridge input unit 211, a collection box 213, a holder 215, a solenoid (217), a vibrator 219, a motor 221, a pump 223, a fixed amount cup 225, an induction tube 227, a communication unit 229, and a control unit 231. FIG. 4 is an internal cross-sectional view of the housing of the urine test device for toilet according to an embodiment of the present invention, and the housing 200 will be described below with reference to FIG. 4.

The sensor unit 201 may include at least one of an insertion sensor 201a, an auxiliary insertion sensor 201b, an image sensor 201c, a removal sensor 201d, a color discrimination sensor 201e, an auxiliary removal sensor 201f, a temperature sensor 201g, humidity sensor 201h and flip sensor 201i.

Referring to FIG. 4, the insertion sensor 201a can sense (judge) whether the beginning part of the urine test strip 310 of the urine test cartridge (not shown) is inserted. The auxiliary insertion sensor 201b can sense (judge) whether the urine test strip 310 has been moved well to the urine collection area. At this time, the insertion sensor 201a and the auxiliary insertion sensor 201b may use a reflection method or an interrupt method of photodiode, but any other sensor that can judge whether the urine test strip 310 is inserted can be used. The urine collection area means the test area (area where elements or enzymes are located) of the urine test strip 310, and may vary depending on the pad size of each element and enzyme and the number of elements and enzymes. As an example, if 10 elements are arranged in 5 columns (2 kinds of row elements arranged) and an element is arranged at 2 mm horizontally, 2 mm vertically and an interval of 2 mm, in total, an area of about 24 mm horizontally and about 10 mm vertically is set as a urine collection area.

The image sensor 201c can recognize the information of the urine test strip 310 by recognizing the barcode area of the inserted urine test strip 310. At this time, the image sensor 201c may use a 1D CCD (Charged Couple Device) or 2D CMOS (Complementary Metal-Oxide-Semiconductor) sensor, or a 1D CIS (Contact Image Sensor) module, but it is not necessarily limited thereto. In addition, an image sensor with a color function can be used to implement various functions of color or monochrome, but this also is not limited thereto.

The removal sensor (201d) and the auxiliary removal sensor (201f) are sensors for detecting to remove the urine test strip 310 stained with urine and a reflection method or an interrupt method using a photodiode can be used, but any sensor capable of detecting the urine test strip may be used.

The color discrimination sensor 201e can measure the color change by measuring the color of the element or enzyme changed by reacting with urine on the urine test strip 310 stained with urine. At this time, the color discrimination sensor 201e can use a 1D CCD (Charged Couple Device) or 2D CMOS (Complementary Metal-Oxide-Semiconductor) sensor, or 1D CIS (Contact Image Sensor), or a color photodiode method sensor made of a single cell. But it is not necessarily limited to this, and any sensor capable of discriminating color can be used. However, when using a color sensor consisting of a single cell, one or more elements or enzymes may be placed in one column of the urine test strip 310, so the number of sensors corresponding to the number of elements or enzymes must be used. In one embodiment, it is generally desirable to arrange two or three elements or enzymes per a row on the urine test strip 310, so, up to three color sensors consisting of single-cell per row should be used. In addition, the color discrimination sensor 201e may be located in a higher area than the urine test strip 310.

When collecting urine using the valve method, the temperature sensor (201g) and humidity sensor (201h) are placed near the induction pipe 227 and can detect the temperature and humidity of the urine collected in the induction pipe 227.

When collecting urine using the fixed amount cup method, the flip sensor 201i is placed near the fixed amount cup 225 and can sense (judge) whether the fixed amount cup 225 has been overturned. In one embodiment, if the fixed amount cup 225 contains urine and is overturned by a seesaw principle, the flip sensor 201i can detect whether it has been overturned. At this time, the flip sensor 201i can use sensor of reflection method or interrupt method of photodiode, but all sensors that can detect whether the cup has been overturned can be used.

When collecting urine using a valve method, the valve 203 can be opened and closed by the control unit 231 and control the discharge of urine when a certain condition is met. Said certain condition may be a case in which the temperature sensor 201g and the humidity sensor 201h match with the average urine discharge temperature and humidity of a person, or may be a case in which the temperature and humidity change suddenly, but is not necessarily limited thereto. In addition, the valve 203 has several small holes formed therein so that urine can be discharged only at the elemental area of the urine test strip 310 and the holes can be opened and closed, but are not necessarily limited thereto.

The roller 205 can be rotated by a motor 221 and refers to a device that can move the urine test strip 310 within the housing 300.

The cutter 207 can separate sectors of the urine test strip 310.

The lifter 209 can lift and remove the protective tape of the urine test strip 310, and can lift it through a solenoid in the lifter 209 or based on another mechanical transfer device. At this time, the protective tape removed by the lifter 209 may be stored in a separate space, or may be discharged into the toilet because it is made of a water-soluble material that dissolves in water.

The cartridge input unit 211 can receive a urine test cartridge (not shown). The urine test cartridge (not shown) may consist of a urine test strip 310 and a reel around which the urine test strip 310 is wound. In this case, the urine test strip 310 may be in the form of several strips connected. In addition, the urine test strip 310 may be equipped with a protective tape to protect it from other foreign substances, but is not necessarily limited to this. A urine test cartridge (not shown) is inserted into the cartridge input unit 211, and only the urine test strip 310 is inserted into the housing 200. And the reel can be stored in a storage box (not shown) next to the cartridge input unit 211. The storage box (not shown) may be attached to the toilet with it inclined toward the outside of the toilet so that the urine test strip 310 can be easily released from the reel, but it is not necessarily limited to this. The reel of the urine test cartridge (not shown) may have a fixing hole in the middle so that it can rotate well in the storage box (not shown), and there is a mechanism that holds the fixing hole so that the reel can rotate without generating noise when it rotates in the storage box.

The collection box 213 is a device that can collect the urine test strip 310 which was used in the urine test.

The holder 215 is a device that can fix the housing 200 inside the toilet.

The solenoid 217 is a device that can adjust the locking state of the fixed amount cup 225 when collecting urine using the fixed amount cup method.

The vibrator 219 is a device for shaking the fixed amount cup 225 to remove residues from the fixed amount cup 225 before urine collection or after completing the urine test when collecting urine using the fixed amount cup method. The vibrator 219 shakes the fixed amount cup 225 to discharge the residue, thereby reducing the error between the previous urine test and subsequent urine test.

The motor 221 can move the roller 205 to move the urine test strip 310. At this time, the motor 221 may be either DC or Stepping motor, but it is not necessarily limited thereto.

The pump 223 is connected to the water in the water tank under the toilet and can draw up water and discharge water into the housing 200 when it is in at least one of the urine test completion mode and cleaning start mode.

The fixed amount cup (225) is a cup that can collect urine and when a certain amount of urine is contained, it is overturned by a seesaw principle due to its weight, allowing the urine to drop onto the urine test strip (310).

The induction pipe 227 is a funnel-shaped pipe capable of inducing urine to be collected and can collect a certain amount of urine before the valve 203.

The communication unit 229 can communicate with each component of the input/output unit 100, the user terminal 500 and the housing 200 through a network. The networks may include at least one or more of 3GPP (3rd Generation Partnership Project) network, LTE (Long Term Evolution) network, 5G network, WIMAX (World Interoperability for Microwave Access) network, wired and wireless Internet, LAN (Local Area Network), Wireless LAN (Wireless Area Network), WAN (Wide Area Network), PAN (Personal Area Network), Bluetooth network, WI-FI network, NFC (Near Field Communication) network, satellite broadcasting network, analog broadcasting network, and DMB (Digital Multimedia Broadcasting) network, but it is not necessarily limited to this. In Addition, the communication unit 220 may use a low-power WIFI module or Bluetooth Low Energy (BLE) Bluetooth Modules, IOT cellular module or Zigbee communication in order to use low power.

The control unit 231 receives at least one mode of urine test start and cleaning start from the input/output unit 100, and may control at least one of the movements of the urine test strip 310 and the control of the pump 223, the control of the opening and closing of the valve 203, the control of overturning of the fixed amount cup 225 through the solenoid 217, the control of the cutter 207 for separating the sectors of the urine test strip 310 in the correct position and the control of lifter 209 for removing the protective tape of the urine test strip 310. The control unit 231 can control each component of the housing 200 and can control each component of the housing 200 according to the communication received from the input/output unit 100.

The user terminal 500 refers to a terminal used by a user who installed the urine test device 10 for toilet on the toilet to test urine, and can be driven through an application for urine test. At this time, the terminal is a device interworking with the urine test device 10 for toilet through a network and for example, may be all types of wireless communication apparatus such as a smartphone, smart pad, tablet PC and the like, PCS (Personal Communication System), GSM (Global System for Mobile communication), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access) and WiBro (Wireless Broadband Internet) terminal, and fixed terminal such as desktop computer and smart TV, but it is not necessarily limited thereto.

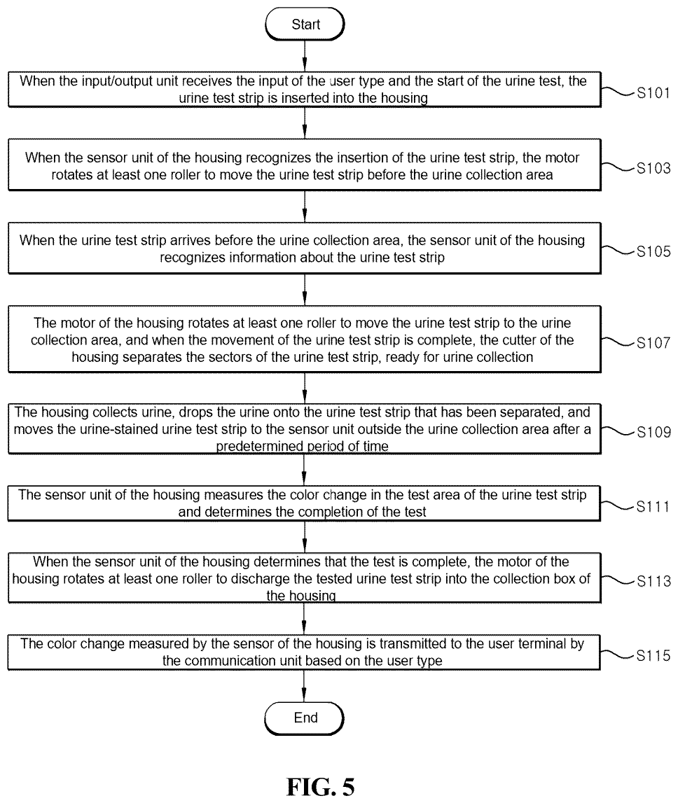

FIG. 5 is a flowchart for explaining the control method of the urine test device for toilet according to an embodiment of the present invention.

Referring to FIG. 5, if the user type and the start of the urine test are input into the input/output unit 100, the urine test strip 310 is inserted into the housing 200 (S101). If the user type is input into the input/output unit 100 from the user, the input/output unit 100 judges that the urine test has started even without a separate input, and can notify the user terminal 500 of the start of the urine test. If the sensor unit 201 of the housing 200 recognizes the insertion of the urine test strip 310, the motor 221 rotates at least one or more rollers 205 to move the urine test strip 310 to before the urine collection area (S103). At this time, the sensor unit 201 of the housing 200 can recognize whether the urine test strip 310 is inserted through the insertion sensor 201a. The auxiliary insertion sensor 201b judges whether the urine test strip 310 has been inserted before the urine collection area, and when the urine test strip 310 arrives just before the urine collection area, the motor 221 stops the rotation of the roller 205.

When the urine test strip 310 arrives before the urine collection area, the sensor unit 201 of the housing 200 recognizes information about the urine test strip 310 (S105). The urine test strip 310 has a barcode area, and the barcode area may contain information about the urine test strip 310. The barcode area is not limited to a barcode and may include information such as OCR in the form of letters. The barcode or OCR may include at least one of the type, date of manufacture and effective date of the urine test strip 310. The image sensor 201c of the sensor unit 201 recognizes the barcode area of the urine test strip 310 to grasp information about the urine test strip 310.

The motor 221 of the housing 200 rotates at least one or more rollers 205 to move the urine test strip 310 to the urine collection area, and when the movement of the urine test strip 310 is completed, the cutter 207 of the housing 200 separates the sectors of the urine test strip 310 to complete preparation for urine collection (S107). The urine test strip 310 used in the present invention is a urine test strip 310 included in a urine test cartridge (not shown), and has a form which several strips used once are connected. Therefore, in order to perform urine test, the connected strips must be separated into sectors by using a cutter 207. When using individual urine test strip without using the urine test strip 310 included in the urine test cartridge (not shown), the process of separating the sectors with the cutter 207 can be omitted. In addition, before separating the sectors, the lifter 209 can lift and remove the protective tape of the urine test strip 310.

The housing 200 collects urine and drops the urine on the separated urine test strip 310, and if a certain time elapses, the urine test strip 310 stained with urine is moved to the sensor unit 201 outside the urine collection area (S109). Since the time required to the color change of the urine test strip 310 varies depending on the element and enzyme of the urine test strip 310, a certain time can be determined based on the maximum time required, but it is not necessarily limited to this. In one embodiment, glucose, which is one of the test elements and enzymes of the urine test strip 310, is recommended to be measured after 30 seconds, so the urine test strip 310 can be moved after 30 seconds. At this time, if it is desirable to measure one of the test elements and enzymes of the urine test strip 310 after 60 seconds, the urine test strip can be moved after 60 seconds. Outside the urine collection area, a removal sensor 201d, an auxiliary removal sensor 201f and a color discrimination sensor 201e may be located. The sensor unit 201 of the housing 200 measures the color change in the test area of the urine test strip 310 and judges the completion of the test (S111). When the color discrimination sensor 201e of the sensor unit 201 completes color measurement for all enzymes and elements of the urine test strip 310, it can judge that the test is completed.

If the sensor unit 201 of the housing 200 judges that the test is completed, the motor 221 of the housing 200 rotates at least one or more rollers 205 to discharge the test-completed urine test strip 310 into the collection box 213 of the housing 200 (S113). When urine is collected using the valve method, for the next use of the urine test device 10 for toilet, the control unit 231 may reopen the valve 203 after the urine test is completed to discharge the remaining urine into the toilet. If the discharge of residual urine is completed, the control unit 231 can close the valve 203 again and transmit the message to the input/output unit 100 to convert to an energy saving mode (sleep mode). If urine was collected using the fixed amount cup method after the urine test is completed, the control unit 231 can transmit the message to the input/output unit 100 to convert to an energy saving mode (sleep mode). The communication unit 229 transmits the color change measured by the sensor unit 201 of the housing 200 to the user terminal 500 based on the user type (S115).

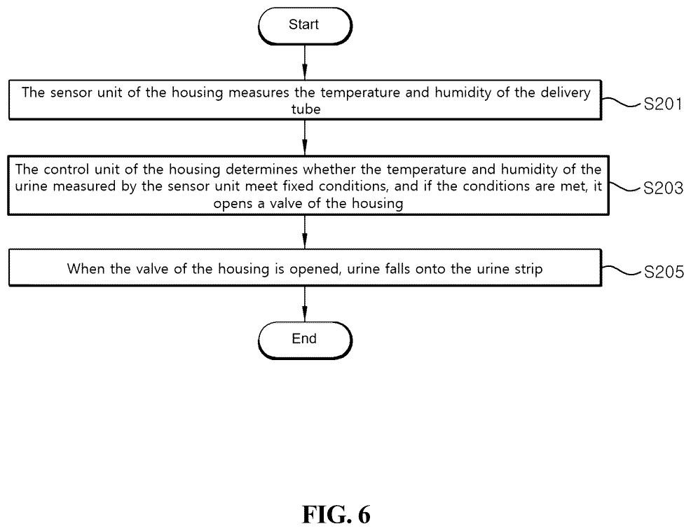

FIG. 6 is a flowchart illustrating the valve method urine collection method of the urine test device for toilet according to an embodiment of the present invention.

Referring to FIG. 6, the sensor unit 201 of the housing 200 measures the temperature and the humidity of the induction pipe 227 (S201). The temperature sensor 201g and the humidity sensor 201h of the sensor unit 201 can measure the temperature and the humidity of urine collected in the guide tube 227. The control unit 231 of the housing 200 judges whether the temperature and humidity of urine measured by the sensor unit 201 meet a certain condition, and if the condition is met, opens the valve 203 of the housing 200 (S203). The valve 203 is always closed usually (when not performing urine test), and may be opened if a certain condition is met when urine test is performed. Said certain condition may be a case in which the temperature sensor 201g and the humidity sensor 201h match with the average urine discharge temperature and humidity of a person, or may be a case in which the temperature and humidity change suddenly, but it is not necessarily limited thereto. In one embodiment, if the temperature and humidity of urine collected in the induction tube 227 and measured by the sensor unit 201 is 36° C. and the humidity is 95%, the control unit 231 judges that this corresponds to a certain condition and can open the valve 203. The control unit 231 may open the valve 203 and close the valve 203 after a certain time. In this case, a certain time may be 1 second, but it is not necessarily limited thereto. When the valve 203 of the housing 200 is opened, urine falls on the urine test strip 310 (S205).

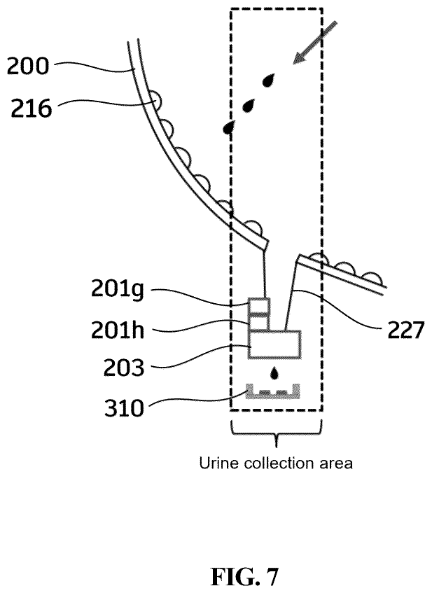

FIG. 7 is a cross-sectional view of the inside of the housing for explaining the valve method urine collection method of the urine test device for toilet according to an embodiment of the present invention.

Referring to FIG. 7, urine touches the outer wall of the housing 200 and collects in the induction tube 227. If urine collects in the induction pipe 227, the temperature sensor 201g and the humidity sensor 201h sense (measure) the temperature and humidity, and when a certain condition is met, the valve 203 opens. If the valve 203 opens, urine is discharged onto the urine test strip 310. After a certain time, the valve 203 is closed, and when the urine test is completed, the valve 203 is opened again to discharge residual urine into the toilet. When the residual urine discharge is completed, the valve 203 can be cleaned by drawing up water from the water tank of the toilet through the pump 223. When cleaning is completed, the valve 203 is closed and converted to energy saving mode (sleep mode).

The outer wall of the housing 200 refers to the area facing the user's urine. The user's urine hits the outer wall of the housing 200 and collects in the guide pipe 227. At this time, protrusion 216 can be installed on the outer wall of the housing 200 to prevent the urine from scattering. The protrusion 216 may be made of a rubber material to maximize the anti-scattering effect, but it is not necessarily limited thereto.

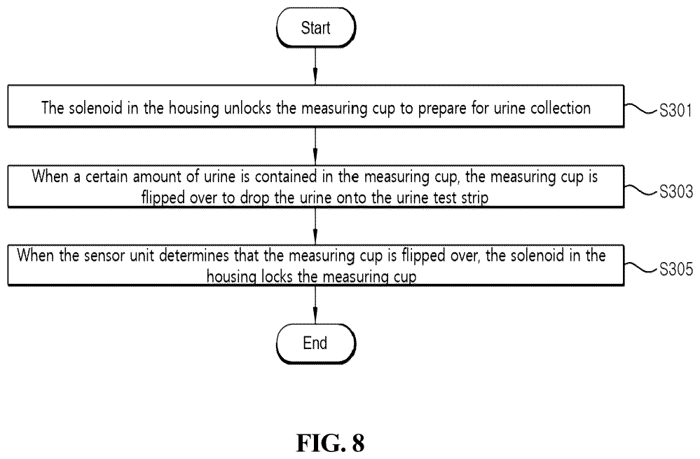

FIG. 8 is a flowchart for explaining the urine collection method using the fixed amount cup method of the urine test device for toilet according to an embodiment of the present invention.

Referring to FIG. 8, if the solenoid 217 of the housing 200 unlocks the fixed amount cup 225, the fixed amount cup 225 rotates so that the upper part can receive urine (S301). Before unlocking the fixed amount cup 225, the fixed amount cup 225 can be shaken using the vibrator 219 to remove moisture or foreign substances. When the fixed amount cup 225 is unlocked, the fixed amount cup 225 is ready to collect the falling urine. When preparations for urine collection are completed, the input/output unit 100 may be notified that preparations for urine collection are complete. When a certain amount of urine is contained in the fixed amount cup 225, the fixed amount cup 225 of the housing 200 is overturned by a seesaw principle due to the weight, and the urine falls on the urine test strip 310 (S303). At this time, by recognizing the signal from the flip sensor 201i, the solenoid 217 of the housing 200 returns the fixed amount cup 225 to its original position and locks it so that the fixed amount cup 225 cannot receive urine again (S305). In this case, the original position may mean an inverted state in which urine cannot be collected, but it is not necessarily limited thereto.

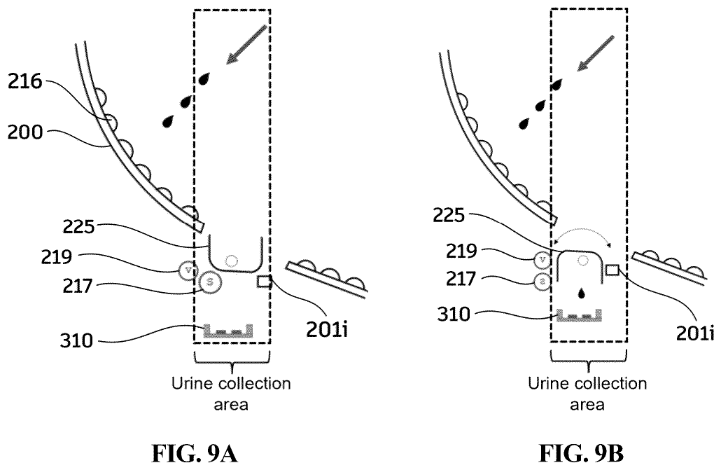

FIG. 9 is a cross-sectional view of the inside of the housing for explaining the urine collection method using a fixed amount cup method of the urine test device for toilet according to an embodiment of the present invention.

The fixed amount cup 225 preferably has a rectangular shape similar to the size of the urine collection area so that it can wet the urine collection area at once when viewed from above, and the rotation axis is aligned side by side in the direction of progress of the urine test strip 310, but the center is made asymmetrical. Asymmetry means that the side where urine flows along the outer wall of the housing 200 is long and the opposite side is short, and a weight can be placed on the short side in order to maintain parallelism when there is no urine in the fixed amount cup 225, but it is not necessarily limited to this.

Referring to FIG. 9(a), it shows a state that the fixed amount cup 225 is unlocked and dripping urine can be collected. At this time, urine can fall into the fixed amount cup 225 along the outer wall of the housing 200. Referring to FIG. 9(b), it shows a state that the fixed amount cup 225 is overturned and urine is discharged onto the urine test strip 310. If a certain amount of urine falls on the urine test strip 310, the solenoid 217 of the housing 200 can lock the fixed amount cup 225 that has returned in an overturned state, and the remaining urine after being stained on the urine test strip 310 flows down into the toilet. The flip sensor 201i can check whether the fixed amount cup 225 has been properly overturned.

The outer wall of the housing 200 refers to the area facing the user's urine. The user's urine hits the outer wall of the housing 200 and collects in the fixed amount cup 225. At this time, a protrusion 216 can be installed on the outer wall of the housing 200 to prevent the urine from scattering. The protrusions 216 may be made of a rubber material to maximize the anti-scattering effect, but it is not necessarily limited thereto.

The invention has been described with reference to the embodiments shown in the drawings, but these are merely illustrative, and those skilled in the art will understand that various modifications and other equivalent embodiments are possible therefrom. Therefore, the true technical protection scope of the present invention should be determined by the technical spirit of the attached registration claims

[DESCRIPTION OF DRAWING NUMBER]

| | |
|---|---|
| 10; urine testing device for toilet | |
| 100; input/output unit | 110; charging module |
| 120; mode selection module | 130; display module |
| 140; communication module | 150; control module |
| 200; housing | 201; sensor unit |
| 203; valve | 205; roller |
| 207; cutter | 209; lifter |
| 211; cartridge input unit | 213; collection box |
| 215; holder | 216: protrusion |
| 217; solenoid | |
| 219; vibrator | 221; motor |
| 223; pump | 225; fixed amount cup |
| 227; induction tube | 229; communication unit |
| 231; control unit | 500; user terminal |

What is claimed is:

1. A urine test device for toilet, comprising:
an input/output unit installed outside the toilet; and
a housing installed inside the toilet and capable of testing urine by staining a urine test strip with urine,
wherein said housing receives the urine test strip and moves the urine test strip to the urine collection area through at least one or more motors and rollers, and can transmit the measurement results to the user terminal after measuring the color change of the urine test strip stained with urine through at least one or more sensors, and
said housing comprises,
a sensor unit that recognizes the barcode of the urine test strip, recognizes whether the urine test strip is inserted and measures the color change of the urine test strip stained with urine;
a cutter that separates the sectors of the urine test strip;
a lifter that removes the protective tape of the urine test strip;
a collection box that collects the urine test strip used in the urine test; and
a pump for performing a cleaning start mode.

2. The urine test device for toilet according to claim 1,
wherein said input/output unit comprises:
a mode selection module that receives the input of at least one mode of user type, user registration, urine test start and cleaning start;
a display module that displays at least one of whether a urine test cartridge is inserted, the remaining amount of the urine test strip, the presence or absence of a malfunction of the urine test device for toilet and the presence or absence of operation of the urine test device for toilet; and
a charging module that manages the charging state of the urine test device for toilet.

3. The urine test device for toilet according to claim 1,
said housing comprises:
an induction tube for collecting urine;
a valve that opens and closes by a control unit when a certain condition is met; and
a control unit that controls the opening and closing of the valve.

4. The urine test device for toilet according to claim 1,
said housing comprises:
a fixed amount cup for collecting urine; and
a solenoid that controls the locking state of said fixed amount cup.

5. The urine test device for toilet according to claim 1,
wherein a mode selection module recognizes the sound of water going down in the toilet using a sound sensor or a microphone, and informs the housing of the cleaning start mode after the water in the toilet has gone down.
